(19) United States
(12) Patent Application Publication
(10) Pub. No.: US 2025/0295325 A1
(43) Pub. Date: Sep. 25, 2025

(54) INTELLIGENCE CAPSULE STRUCTURE

(71) Applicant: Universal Scientific Industrial (Shanghai) Co., Ltd., Shanghai (CN)

(72) Inventors: CHUNG-PING CHANG, Shanghai (CN); HSIU LIEH CHENG, Shanghai (CN); KUAN-YING SHEN, Shanghai (CN)

(21) Appl. No.: 19/021,210

(22) Filed: Jan. 15, 2025

(30) Foreign Application Priority Data
Mar. 25, 2024 (CN) 202410346962.6

(51) Int. Cl. A61B 5/07; A61B 1/00; A61B 1/04; A61B 5/00

(52) U.S. Cl. CPC A61B 5/073; A61B 1/00032; A61B 1/0011; A61B 1/041; A61B 5/0062; A61B 5/6861; A61B 2560/0214; A61B 2560/0462; A61B 2562/0247; A61B 2562/0271; A61B 2562/162; A61B 2562/166

(57) ABSTRACT

The intelligence capsule structure includes a shell, a detecting module and a power supplying module. The shell has an axial direction. The detecting module is disposed in the shell and includes at least two substrates and at least one sensor. The at least two substrates are disposed along the axial direction. The at least one sensor is disposed on the at least two substrates, and is configured to capture at least one pathophysiological data. The power supplying module electrically is connected to the detecting module, and is disposed in the shell along the direction perpendicular to the axial direction.

### RELATED APPLICATIONS

This application claims priority to China Application Serial Number 202410346962.6, filed Mar. 25, 2024, which is herein incorporated by reference.

### BACKGROUND

### Technical Field

The present disclosure relates to a monitoring structure. More particularly, the present disclosure relates to an intelligence capsule structure for monitoring the human body.

### Description of Related Art

After the intelligence capsule is swallowed by the patient, it enters the body and moves autonomously or passively along the gastrointestinal tract to capture the in vivo information which is difficult to obtain. After completing the detection, the intelligence capsule is easily excreted through the digestive system and provides the doctor for diagnosis based on the in vivo information of the patient. With the increase in the number of the patients with chronic diseases and the increasing demand for precision medicine, the market of the intelligence capsule is placed in the stage in booming development, and the medical device manufacturers, the pharmaceutical companies and the top academic teams all have dedicated to research and development.

The existing intelligence capsule includes a shell, a rigid-flex board, a plurality of sensing elements and button batteries. First, set the sensing element on the two hard boards of the rigid-flex board, and connect the two hard boards with the soft board of the rigid-flex board. After sequentially placing one hard board and the button battery into the shell, put the other hard board into the shell so that the button battery is located between the two hard boards, and the positive pole and negative pole of the button battery are connected to the two hard boards, respectively.

However, the existing intelligence capsule is complicated and time-consuming to assemble, and the hard board of the rigid-flex board is not easy to produce the fine-line. Therefore, the volume of the intelligence capsule cannot be further reduced. In view of this, developing an intelligence capsule structure which is easy to assemble and with small size has become the goal of the relevant industries.

### SUMMARY

The present disclosure provides an intelligence capsule structure. The intelligence capsule structure includes a shell, a detecting module and a power supplying module. The shell has an axial direction. The detecting module is disposed in the shell and includes at least two substrates and at least one sensor. The at least two substrates are disposed along the axial direction. The at least one sensor is disposed on the at least two substrates, and is configured to capture at least one pathophysiological data. The power supplying module is electrically connected to the detecting module, and is disposed in the shell along the direction perpendicular to the axial direction.

### BRIEF DESCRIPTION OF THE DRAWINGS

The present disclosure can be more fully understood by reading the following detailed description of the embodiment, with reference made to the accompanying drawings as follows:

FIG. 1 is a three-dimensional schematic view of an intelligence capsule structure according to one embodiment of the present disclosure.

FIG. 2 is an exploded view of the intelligence capsule structure according to the embodiment of FIG. 1.

FIG. 3 is a block flow chart of an assembly method of the intelligence capsule structure in FIG. 1 of the present disclosure.

FIG. 4A is a schematic view of the assembly method in FIG. 3, which is for mounting the substrates on a single side or double sides.

FIG. 4B is a schematic view of the assembly method in FIG. 3, which is for implanting the copper pillars on the substrates.

FIG. 4C is a schematic view of the assembly method in FIG. 3, which is for connecting the substrates and the conducting sheets.

FIG. 4D is a schematic view of the assembly method in FIG. 3, which is for placing the detecting module and the conducting sheets into a first shell portion.

FIG. 4E is a schematic view of the assembly method in FIG. 3, which is for placing a power supplying module into the first shell portion.

FIG. 4F is a schematic view of the assembly method in FIG. 3, which is for locking the second shell portion.

### DETAILED DESCRIPTION

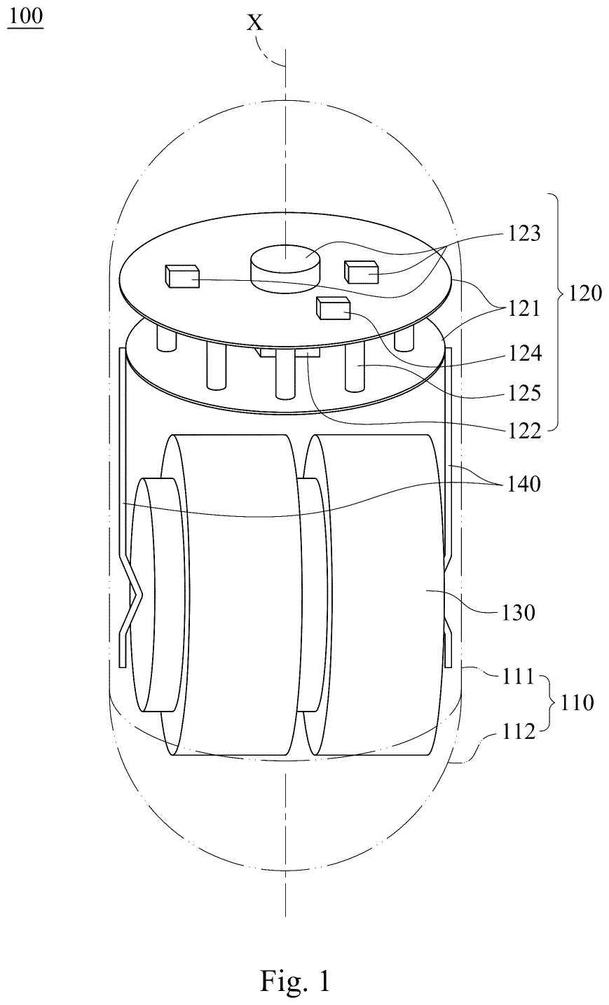

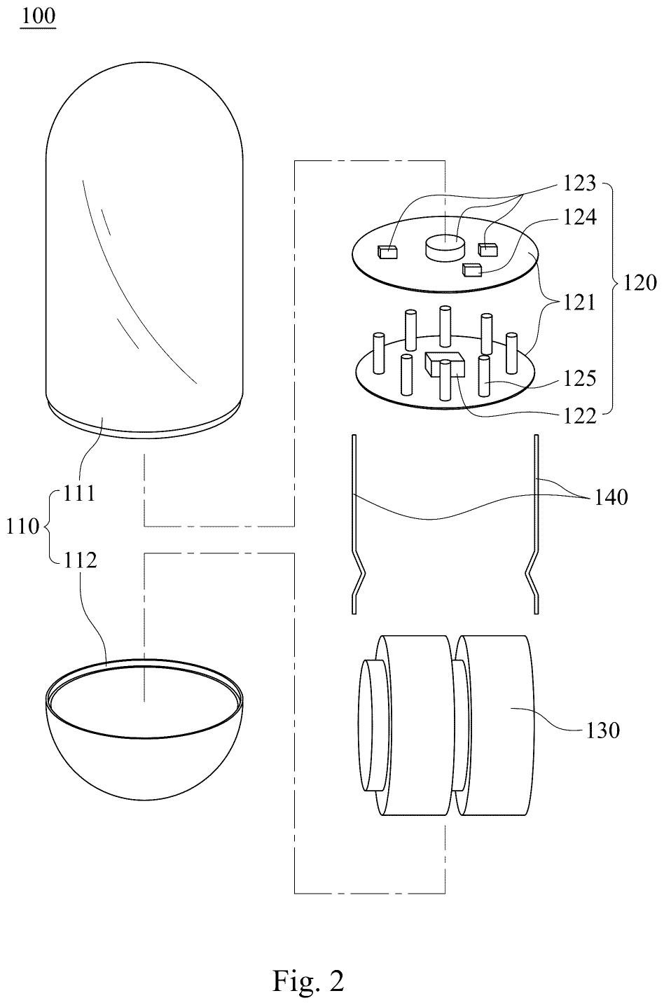

FIG. 1 is a three-dimensional schematic view of an intelligence capsule structure 100 according to one embodiment of the present disclosure. FIG. 2 is an exploded view of the intelligence capsule structure 100 according to the embodiment of FIG. 1. The intelligence capsule structure 100 is configured to capture the in vivo information of the user and assist the doctor in diagnosis by entering the user's body after swallowing, and excreting through the digestive system after completing the detection.

As shown in FIG. 1, the intelligence capsule structure 100 includes a shell 110, a detecting module 120 and a power supplying module 130, the detecting module 120 and the power supplying module 130 are disposed in the shell 110, the detecting module 120 is electrically connected to the power supplying module 130. In addition, the intelligence capsule structure 100 can further include two conducting sheets 140, the two conducting sheets 140 are disposed in the shell 110, and are configured to electrically connect the detecting module 120 and the power supplying module 130, but the present disclosure is not limited thereto.

The shell 110 has an axial direction X, the detecting module 120 is disposed along the axial direction X, the power supplying module 130 is located at one end of the detecting module 120 and disposed along the direction perpendicular to the axial direction X. The two conducting sheets 140 are disposed along the axial direction X and are located at two ends of the power supplying module 130, respectively. One end of each of the two conducting sheets 140 is connected to the detecting module 120, and the other end of each of the two conducting sheets 140 is connected to the power supplying module 130.

The shell 110 includes a first shell portion 111 and a second shell portion 112, the first shell portion 111 is detachably connected to the second shell portion 112 along the axial direction X. A length of the first shell portion 111 is greater than a length of the second shell portion 112. In the embodiment of the FIG. 1, a diameter of the shell 110 is less than 16 mm; the material of the shell 110 is plastic, but the present disclosure is not limited thereto.

In other embodiments, the shell can be wrapped around the outside of the detecting module, the power supplying module and the conducting sheets through the insert molding to form the intelligence capsule structure.

The detecting module 120 includes at least two substrates 121, a controller 122, at least one sensor 123 and a memory 124. The at least two substrates 121 are disposed along the axial direction X, the controller 122, the at least one sensor 123 and the memory 124 are disposed on the at least two substrates 121. The controller 122 is electrically connected to the at least one sensor 123 and the memory 124, the memory 124 is electrically connected to the at least one sensor 123. It should be noted that, in other embodiments, the memory can also be built-in the controller.

The at least two substrates 121 are configured to provide circuit layout design, and provide the controller 122, the at least one sensor 123 and the memory 124 to be mounted on a single side or double sides of the at least two substrates 121 by surface-mount technology (SMT). Each of the at least two substrates 121 is printed circuit board (PCB). In the embodiment of the FIG. 1, each of the at least two substrates 121 is in a disc-shaped, and a diameter of each of the at least two substrates 121 is between 5 mm and 10 mm, a number of the at least two substrates 121 is two, but the present disclosure is not limited thereto.

Therefore, it is favorable for easily producing the fine-line on the single side or the double sides of the substrates 121, and the controller 122, the sensor 123 and the memory 124 can be mounted and performed circuit layout on the substrates 121 in extremely small size so as to reduce the size of the intelligence capsule structure 100 to make it easier for the user to swallow by using PCB as the substrate 121.

The controller 122 is configured to control the at least one sensor 123 to capture at least one pathophysiological data. In the embodiment of the FIG. 1, the controller 122 can be a microprocessor, but the present disclosure is not limited thereto.

The at least one sensor 123 is configured to capture the at least one pathophysiological data, the at least one sensor 123 is a pressure sensor, a temperature sensor, an image sensor, a pH sensor or a vibration sensor, but the present disclosure is not limited thereto. In the embodiment of the FIG. 1, a number of the at least one sensor 123 is three, which are the pressure sensor, the temperature sensor and the image sensor, and can sense the intra-abdominal hypertension (IAH), the body ambient temperature and the endoscopy images of the user, respectively.

The memory 124 is configured to store the pathophysiological data. In the embodiment of the FIG. 1, the memory 124 can be a Random Access Memory (RAM) or another type of dynamic storage device that can store information and instructions for execution by the controller 122.

The power supplying module 130 is configured to provide the working power for the detecting module 120. The power supplying module 130 includes at least one button battery (its reference numeral is omitted), the at least one button battery is disposed along the direction perpendicular to the axial direction X. In the embodiment of the FIG. 1, a number of the at least one button battery is two.

In addition, the power supplying module 130 can further include a non-conductive battery holder (not shown) to further fix the button batteries and prevent the button batteries from rotating during the movement of the intelligence capsule structure 100 and affecting the sensing effect of the at least one sensor 123. It should be noted that, in other embodiments, the shell can be made by insert molding, when the shell wraps around the button batteries, it also has the effect of fixing the button batteries.

The two conducting sheets 140 are configured to electrically connect the detecting module 120 and the power supplying module 130. One end of one of the two conducting sheets 140 is connected to the at least two substrates 121, and the other end is connected to the positive pole of one of the button batteries. One end of the other one of the two conducting sheets 140 is connected to the at least two substrates 121, and the other end is connected to the negative pole of the other one of the button batteries. Therefore, by utilizing the two conducting sheets 140, it is favorable for sequentially placing the detecting module 120 and the power supplying module 130 into the shell 110 during the assembly of the intelligent capsule structure 100 so as to ensure the button battery is electrically connected to the detecting module 120 to supply power.

As shown in FIG. 1 and FIG. 2, the detecting module 120 further includes a plurality of copper pillars 125, the plurality of copper pillars 125 are disposed between the at least two substrates 121, and are disposed around the surface periphery of the at least two substrates 121. The plurality of copper pillars 125 are configured to electrically connect the at least two substrates 121. In the embodiment of the FIG. 1, a diameter of each of the plurality of copper pillars 125 is between 0.1 mm and 0.5 mm, but the present disclosure is not limited thereto. Further, a number of the plurality of copper pillars 125 is set according to the complexity of the circuit, and the number of the copper pillars 125 is between 10 and 78, but the present disclosure is not limited thereto.

In detail, the controller 122, the sensor 123 and the memory 124 can be laid out on the at least two substrates 121 according to the requirements. The information output and input requirements of the sensor 123 can be met by electrically connecting the at least two substrates 121 through the copper pillars 125. Therefore, it is favorable for further arranging the components densely so as to greatly reduce the size of the intelligence capsule structure 100.

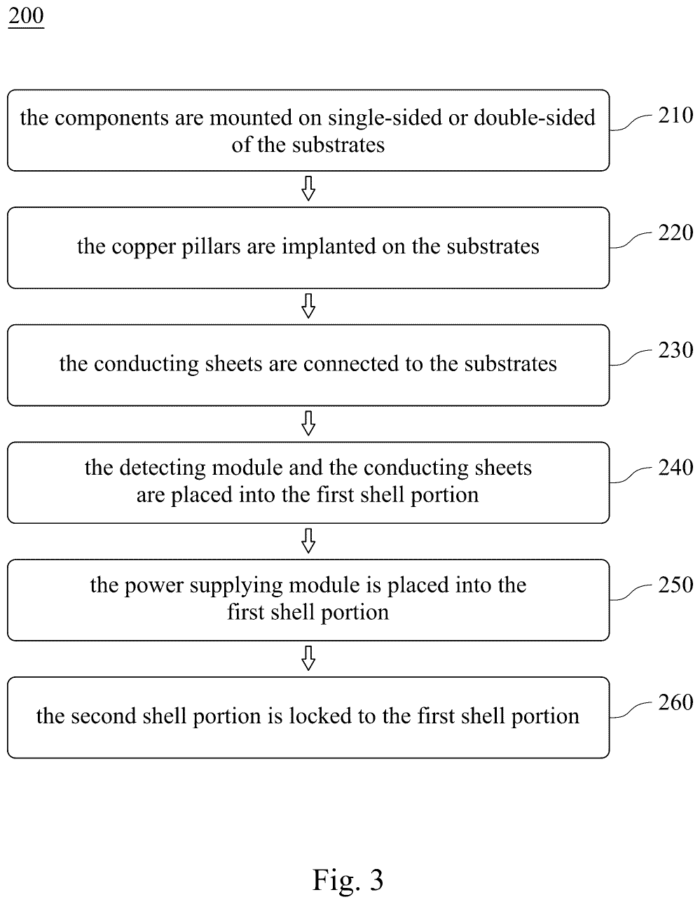

FIG. 3 is a block flow chart of an assembly method 200 of the intelligence capsule structure 100 in FIG. 1 of the present disclosure. According to the above embodiment, the assembly method 200 of the intelligence capsule structure 100 is proposed below in detail, as shown in FIG. 1 to FIG. 3, the assembly method 200 includes step 210, step 220, step 230, step 240, step 250 and step 260.

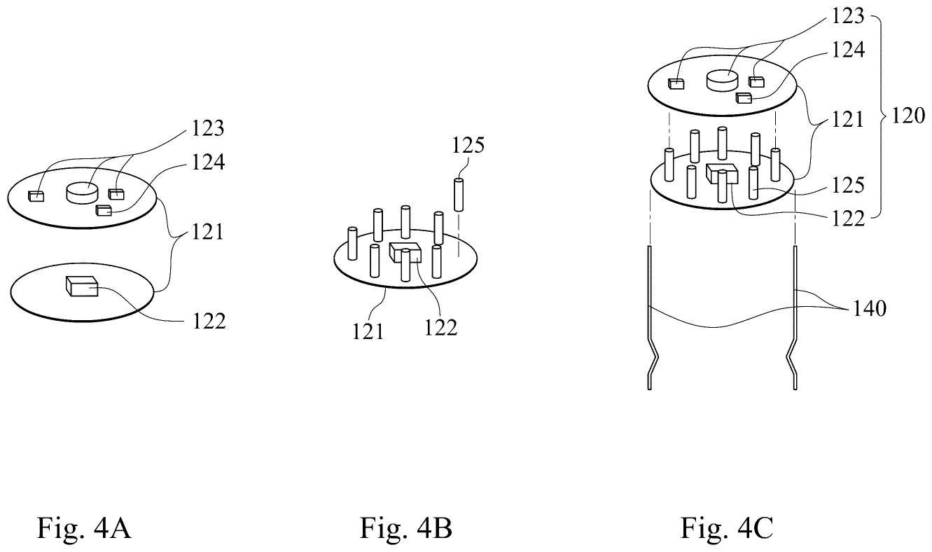

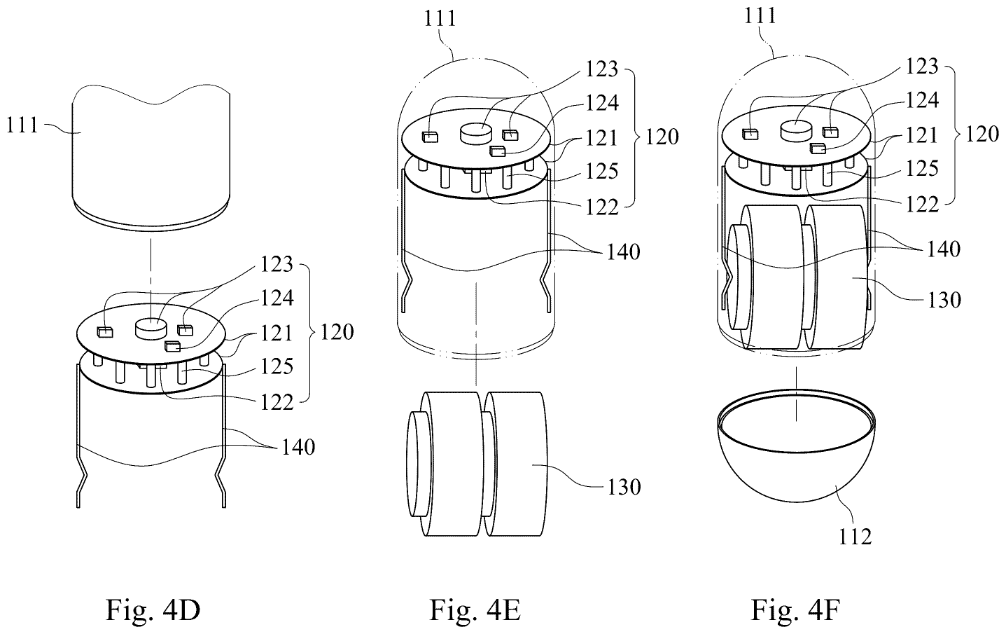

FIG. 4A is a schematic view of the assembly method 200 in FIG. 3, which is for mounting the substrates 121 on the single side or the double sides. As shown in FIG. 3 and FIG. 4A, in step 210, the components are mounted on the single side or the double sides of the substrates 121 through SMT, and the controller 122, the sensor 123 and the memory 124 are soldered to the substrates 121 which has been routed as required.

FIG. 4B is a schematic view of the assembly method 200 in FIG. 3, which is for implanting the copper pillars 125 on the substrates 121. As shown in FIG. 3 and FIG. 4B, in step 220, the copper pillars 125 are implanted on one of the substrates 121, and are disposed around the surface periphery of the one of the substrates 121.

FIG. 4C is a schematic view of the assembly method 200 in FIG. 3, which is for connecting the substrates 121 and the conducting sheets 140. As shown in FIG. 3 and FIG. 4C, in step 230, the substrates 121 are connected to each other by welding to form the detecting module 120, and the conducting sheets 140 are connected to one of the substrates 121 by welding.

FIG. 4D is a schematic view of the assembly method 200 in FIG. 3, which is for placing the detecting module 120 and the conducting sheets 140 into a first shell portion 111. As shown in FIG. 3 and FIG. 4D, in step 240, the detecting module 120 and the conducting sheets 140 are placed into the first shell portion 111.

FIG. 4E is a schematic view of the assembly method 200 in FIG. 3, which is for placing a power supplying module 130 into the first shell portion 111. As shown in FIG. 3 and FIG. 4E, in step 250, the power supplying module is further placed into the first shell portion 111. One of the conducting sheets 140 is connected to the positive pole of one of the button batteries, and the other one of the conducting sheets 140 is connected to the negative pole of the other one of the button batteries. Therefore, the button battery can electrically connect to the detecting module 120 to supply power after placing the detecting module 120 and the power supplying module 130 into the shell 110, sequencely.

FIG. 4F is a schematic view of the assembly method 200 in FIG. 3, which is for locking the second shell portion 112. As shown in FIG. 3 and FIG. 4F, in step 260, the second shell portion 112 is locked to the first shell portion 111 to complete the assembly of the intelligence capsule structure 100.

In view of the above, the present disclosure has the following advantages. First, it is favorable for assembling the intelligence capsule structure easily and quickly by the configuration of the shell, the detection module and the power supplying module. Second, it is favorable for reducing the size of the intelligence capsule structure and make the intelligence capsule structure easier for the user to swallow by producing the fine-line on the substrates in extremely small size and laying out the controller, the sensor and the memory densely to form the detecting module.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. An intelligence capsule structure, comprising:
a shell having an axial direction;
a detecting module disposed in the shell and comprising:
at least two substrates disposed along the axial direction; and
at least one sensor disposed on the at least two substrates, and configured to capture at least one pathophysiological data; and
a power supplying module electrically connected to the detecting module, and disposed in the shell along the direction perpendicular to the axial direction.

2. The intelligence capsule structure of claim 1, wherein the detecting module further comprises:
a plurality of copper pillars disposed between the at least two substrates, and configured to electrically connect the at least two substrates.

3. The intelligence capsule structure of claim 2, wherein a diameter of each of the plurality of copper pillars is between 0.1 mm and 0.5 mm.

4. The intelligence capsule structure of claim 1, wherein the detecting module further comprises:
a controller disposed on the at least two substrates and electrically connected to the at least one sensor, the controller configured to control the at least one sensor.

5. The intelligence capsule structure of claim 4, wherein the at least one sensor and the controller are disposed on the at least two substrates by surface-mount technology.

6. The intelligence capsule structure of claim 1, wherein each of the at least two substrates is printed circuit board.

7. The intelligence capsule structure of claim 1, wherein each of the at least two substrates is in a disc-shaped, and a diameter of each of the at least two substrates is between 5 mm and 10 mm.

8. The intelligence capsule structure of claim 1, wherein the at least one sensor is a pressure sensor, a temperature sensor, an image sensor, a pH sensor or a vibration sensor.

9. The intelligence capsule structure of claim 1, further comprising:
two conducting sheets disposed in the shell along the axial direction, one end of each of the two conducting sheets connected to the detecting module, and the other end of each of the two conducting sheets connected to the power supplying module.

10. The intelligence capsule structure of claim 1, wherein the shell comprises:
a first shell portion; and
a second shell portion detachably connected to the first shell portion along the axial direction.
